(19) United States
(12) Patent Application Publication
(10) Pub. No.: US 2023/0309479 A1
(43) Pub. Date: Oct. 5, 2023

(54) CULTURING METHOD AND CULTURING DEVICE

(71) Applicant: HONDA MOTOR CO., LTD., Tokyo (JP)

(72) Inventor: Nozomi Shiobara, Wako-shi (JP)

(21) Appl. No.: 18/128,283

(22) Filed: Mar. 30, 2023

(30) Foreign Application Priority Data
Mar. 31, 2022 (JP) 2022-059386

(51) Int. Cl. A01G 33/00; C12M 1/00

(52) U.S. Cl. CPC A01G 33/00; C12M 29/24

(57) ABSTRACT

A culturing device is provided with an accommodation portion that accommodates culturing solution and microalgae. The accommodation portion is inclined with respect to the vertical direction and is supported by a support body. In this state, microalgae are cultured. A gas gathering portion is provided at an upper part of a side wall of the inclined accommodation portion, the outer surface of the side wall facing obliquely upward. The gas produced by the microalgae is gathered by the gas gathering portion.

### CROSS-REFERENCE TO RELATED APPLICATIONS

This application is based upon and claims the benefit of priority from Japanese Patent Application No. 2022-059386 filed on Mar. 31, 2022, the contents of which are incorporated herein by reference.

### BACKGROUND OF THE INVENTION

### Field of the Invention

The present invention relates to a culturing method and a culturing device for culturing microalgae in a culturing solution.

### Description of the Related Art

Efforts aimed at climate change mitigation or impact reduction have continued, and toward the realization thereof, research and development in relation to the reduction of carbon dioxide emissions are being carried out. From this point of view, attention has been focused on microalgae. This is because microalgae consume carbon dioxide through photosynthesis. Therefore, a culturing device for culturing microalgae is anticipated as a device that contributes to the climate change mitigation or impact reduction.

Furthermore, by culturing the microalgae, it is possible to obtain liquid fuel such as ethanol or light oil. In addition, as described in JP H04-018837 B2, it is known that a certain kind of microalgae generates hydrogen gas under a predetermined culture environment. Thus, microalgae make it possible to obtain various resources.

As described in JP 3213454 U, when microalgae are cultured, it is general to supply a culturing gas into an accommodation portion in which microalgae and a culturing solution are accommodated. In this case, the accommodation portion is provided with a fluid passage. The culturing gas is supplied toward the fluid passage.

### SUMMARY OF THE INVENTION

The gas produced by the microalgae is released into the culturing solution. Therefore, the gas and the culturing gas supplied to a guide portion are mixed in the culturing solution. Therefore, in order to obtain the gas as a high-purity gas, a mixed gas of the gas and the culturing gas is collected and then the mixed gas is separated into the gas and the culturing gas. As described above, in the prior art, a complicated operation of purifying the gas produced by the microalgae is required.

An object of the present invention is to solve the above-described problems.

According to one embodiment of the present invention, provided is a culturing method of gathering gas produced by microalgae in a culturing solution, the method comprising culturing the microalgae while guiding, with a guide portion, culturing gas from the bottom of the accommodation portion accommodating the culturing solution and the microalgae toward the liquid surface of the culturing solution, in a state in which an accommodation portion is inclined with respect to the vertical direction, and providing a gas gathering portion at an upper portion of a side wall of the inclined accommodation portion, an outer surface of the side wall facing obliquely upward, and gathering with the gas gathering portion the gas produced by the microalgae being cultured.

According to another embodiment of the present invention, provided is a culturing device that gathers gas produced by microalgae in a culturing solution, the culturing device including an accommodation portion that accommodates the culturing solution and the microalgae, a support body that supports the accommodation portion in a posture inclined with respect to the vertical direction, a gas supply unit that supplies culturing gas to the culturing solution in the accommodation portion, a guide portion that is provided to the accommodation portion and guides the culturing gas from the bottom of the accommodation portion toward the liquid surface of the culturing solution, and a gas gathering portion that is provided at an upper portion of a side wall of the inclined accommodation portion, an outer surface facing obliquely upward, wherein the gas produced by the microalgae being cultured is gathered by the gas gathering portion.

In the present invention, microalgae are cultured in an accommodation portion that is at an angle with respect to the vertical direction. When the microalgae generate gas with the accommodation portion being inclined, the gas (bubble) goes up in the culturing solution toward the side wall, the outer surface of the side wall of the accommodation portion facing obliquely upward. The gas that has contacted the side wall further moves along the side wall toward the liquid surface of the culturing solution. Here, a gas gathering portion is provided at an upper portion of the side wall. Therefore, the gas moving toward the liquid surface can be easily gathered by the gas gathering portion.

A culturing gas is supplied to the culturing solution in the accommodation portion. The culturing gas is guided by a guide portion provided in the accommodation portion and moves from the bottom of the accommodation portion toward the liquid surface of the culturing solution. That is, the culturing gas passes through a path different from the path for the gas generated by the microalgae in the accommodation portion. Therefore, mixing of the gas and the culturing gas is prevented.

For the above reasons, the gas produced by the microalgae can be collected as a high-purity gas. This eliminates the need for processes that separate the collected gas and the culturing gas from each other. That is, a high-purity gas can be easily obtained by a simple procedure.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a schematic system diagram of a culturing device according to an embodiment of the present invention.

FIG. 2 is a schematic side cross-sectional view of the culturing device.

FIG. 3 is an enlarged view of principal parts of FIG. 2.

### DETAILED DESCRIPTION OF THE INVENTION

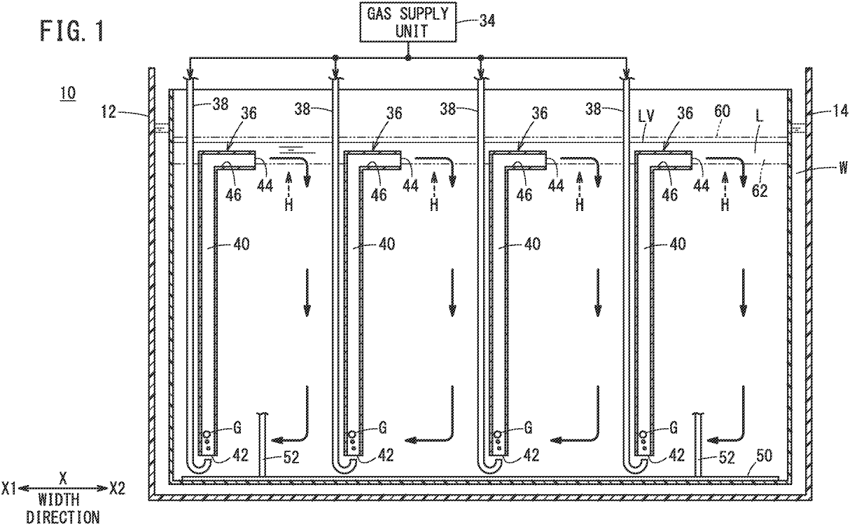

FIG. 1 is a schematic system diagram of a culturing device 10 according to the present embodiment. A culturing device 10 is provided with a water storage portion 12 and an accommodation portion 14 arranged inside the water storage portion 12. Water W is stored in the water storage section 12. The water W is a cooling medium for cooling a culturing solution L in the accommodation portion 14.

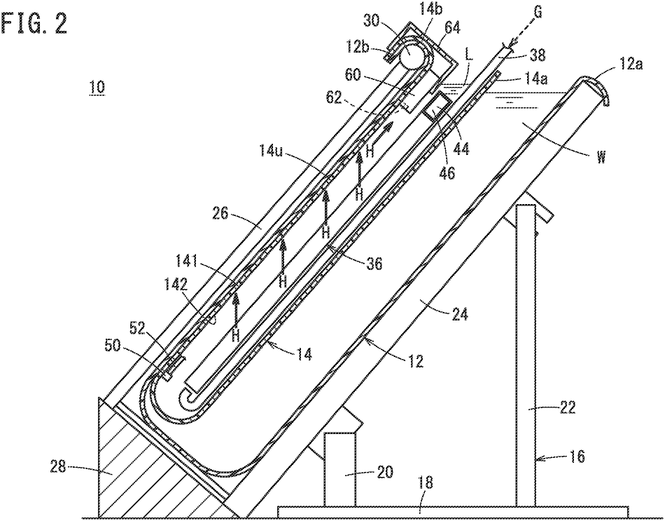

FIG. 2 is a schematic side cross-sectional view of the culturing device 10. The culturing device 10 is provided with a frame 16 for supporting the water storage section 12 and the accommodation portion 14. The frame 16 is a support body for supporting the water storage section 12 and the accommodation portion 14 taking inclined postures with respect to the vertical direction. Specifically, the frame 16 has a pedestal portion 18, a first leg portion 20, a second leg portion 22, a first inclined plate 24, and a second inclined plate 26. The first leg portion 20 and the second leg portion 22 extend vertically upward from the pedestal portion 18. The length (height) of the first leg portion 20 is smaller than the length (height) of the second leg portion 22. The first inclined plate 24 is supported by the first leg portion 20 and the second leg portion 22.

The frame 16 has an inclined base 28 for supporting the first inclined plate 24 and the second inclined plate 26. The first inclined plate 24 is connected to a lower end of an inclined surface of the inclined base 28 so as to be substantially perpendicular thereto. The second inclined plate 26 is connected to an upper end of the inclined surface of the inclined base 28 so as to be substantially perpendicular thereto. Therefore, the first inclined plate 24 and the second inclined plate 26 extend substantially parallel to each other. The angles of the first inclined plate 24 and the second inclined plate 26 with respect to the vertical direction is, for example, 20° to 70°, and preferably 35° to 55°. It should be noted that the first inclined plate 24 and the second inclined plate 26 are formed of transparent/translucent material that allows light to pass through.

The accommodation portion 14 is a bag-like container made of flexible and the transparent/translucent material. As a constituent material of the accommodation portion 14, for example, linear low density polyethylene (LLDPE) can be used. The accommodation portion 14 is formed into a bag shape with a closed lower end by, for example, folding a band-like sheet made of a single piece of flexible material in half and welding both width-direction ends.

In a state in which the water storage section 12 is inclined as shown in FIG. 2, the water storage section 12 has a lower upper end 12a facing the first inclined plate 24 and a higher upper end 12b facing the second inclined plate 26. The lower upper end 12a is folded back to the outer side of the first inclined plate 24 and positioned and fixed to the upper end of the first inclined plate 24. The higher upper end 12b is folded back to the outer side of the second inclined plate 26. A beam portion 30 is provided at an upper end of the second inclined plate 26, and the higher upper end 12b is positioned and fixed to the beam portion 30 via a fixing tool 64 described later.

A culturing solution L and microalgae are contained in the accommodation portion 14. As understood from the explanation, the accommodation portion 14 is a culture tank for culturing microalgae. The culturing solution L is typically water. It is preferable to add phosphorus, nitrogen, potassium, and suchlike to the culturing solution L in advance.

In a state in which the accommodation portion 14 is inclined as shown in FIG. 2, the accommodation portion 14 has a lower upper end 14a facing the first inclined plate 24 and a higher upper end 14b facing the second inclined plate 26. The higher upper end 14b is joined to the higher upper end 12b of the water storage section 12. Accordingly, the higher upper end 14b of the accommodation portion 14 is folded back to the outer side of the second inclined plate 26 together with the higher upper end 12b of the water storage portion 12 and is positioned and fixed to the beam portion 30 via the fixing tool 64.

The accommodation portion 14 has a side wall 14u that has an outer surface 141 facing obliquely upward with respect to the vertical direction. The inclination angle of the side wall 14u of the accommodation portion 14 with respect to the vertical direction is substantially the same as the inclination angles of the first inclined plate 24 and the second inclined plate 26 with respect to the vertical direction. The side wall 14u faces the second inclined plate 26.

As shown in FIG. 1, the culturing device 10 further includes a gas supply unit 34 for supplying the culturing gas G and a plurality of guide portions 36. The gas supply unit 34 is, for example, an air pump. The gas supply unit 34 may be a factory exhaust line. One end of a gas supply pipe 38 is connected to the gas supply unit 34. The other end of the gas supply pipe 38 branches into as many lines as the guide portions 36. The branched gas supply pipe 38 is arranged in the vicinity of each guide portion 36.

As understood from FIG. 1, the guide portion 36 is a hollow body having a substantially inverted L-shape. A substantially inverted L-shaped guide flow path 40 is formed in the hollow interior of the guide portion 36. A suction port 42 is formed at the lower end of the guide portion 36. The downstream end of the gas supply pipe 38 is bent toward the suction port 42. At the upper end of the guide portion 36, a protrusion 44 projecting rightward in FIG. 1 is formed with a discharge port 46.

The plurality of guide portions 36 are arranged at intervals in the width direction of the accommodation portion 14 (direction of arrow X in FIG. 1). The plurality of guide portions 36 cooperate with the culturing gas G supplied from the gas supply unit 34 to cause convection in the culturing solution L in the accommodation portion 14. Specifically, when the culturing gas G is supplied into the guide portion 36 (guide flow path 40), each guide portion 36 draws the culturing solution L into the guide portion 36 and discharges the culturing solution L to the outside of the guide portion 36. In this way, convection of the culturing solution L is generated in the accommodation portion 14. In FIG. 1, convection is shown by solid lines.

Although not shown in detail, the guide portion 36 is supported by the frame 16, for example. Alternatively, the guide portion 36 may be supported by a frame separate from the frame 16. Alternatively, the guide portion 36 may be joined to the inner surface of the accommodation portion 14.

The culturing device 10 further includes a wiper 50 as a sliding member. The wiper 50 extends along the width direction of the culturing device 10 (direction of arrow X in FIG. 1). The wiper 50 is connected to the tips of two rods 52, for example. The two rods 52 are raised or lowered along the side wall 14u by an actuator (not shown). The wiper 50 moves up or down integrally with the two rods 52.

A gas gathering portion 60 is provided at an upper portion (near the higher upper end 14b) of a side wall 14u of the accommodation portion 14. A lower portion of the gas gathering portion 60 is immersed in the culturing solution L. In other words, the lower portion of the gas gathering portion 60 is positioned below the liquid level of the culturing solution L. In the illustrated example, the upper portion of the gas gathering portion 60 is exposed from the culturing solution L, but the entire gas gathering portion 60 may be immersed in the culturing solution L.

A gathering port 62 is formed on the lower surface of the gas gathering portion 60. The gathering port 62 is open in the culturing solution L. The gathering port 62 is positioned below the liquid level of the culturing solution L. The gathering port 62 is positioned below the discharge port 46 formed at the upper end of the guide portion 36. Alternatively, the gathering port 62 is positioned at the same height as the discharge port 46 (the upper end of the guide portion 36).

The gas gathering portion 60 is positioned and fixed to the beam portion 30 via the fixing tool 64. The fixing tool 64 sandwiches the gas gathering portion 60 and the beam portion 30. From the inner side to the outer side of the beam portion 30, the higher upper end 14b of the accommodation portion 14 and the higher upper end 12b of the water storage section 12 are folded back. Therefore, the higher upper end 14b of the accommodation portion 14 and the higher upper end 12b of the water storage portion 12 are positioned and fixed to the beam portion 30 by the fixing tool 64.

In the present embodiment, Chlamydomonas is selected as the microalgae. Chlamydomonas produces hydrogen gas H under a predetermined culturing environment.

Next, a culturing method according to the present embodiment will be described.

First, an operator prepares a culturing solution L whose components have been adjusted. Specifically, the operator prepares a culturing solution L in which sulfur (S) is deficient and nitrogen (N) is deficient. Next, as shown in FIG. 2, the operator stores water W in the water storage section 12 in a state in which the water storage section 12 and the accommodation portion 14 are at an angle. Further, Chlamydomonas and the culturing solution L prepared as described above are stored in the accommodation portion 14.

Next, the operator supplies the culturing gas G to the accommodation portion 14. When the gas supply unit 34 is an air pump, the operator starts the air pump. The air sucked by the air pump contains carbon dioxide. The exhaust gas discharged into the factory exhaust line also contains carbon dioxide. Therefore, the gas supply unit 34 supplies the carbon-dioxide-containing gas as the culturing gas G.

The culturing gas G flowing through the gas supply pipe 38 is distributed to each of the plurality of guide portions 36. As described above, the suction port 42 is formed at the lower end of each guide portion 36. The culturing gas G discharged from the gas supply pipe 38 enters the interior of the guide portion 36 (guide flow path 40) via the suction port 42. At this time, the culturing solution L and the microalgae around the suction port 42 are entrained in the culturing gas G and sucked from the suction port 42. The sucked culturing solution L and the microalgae rise in the guide portion 36 along the guide flow path 40 accompanied by the culturing gas G. In this way, the guide portion 36 guides the culturing gas G, the culturing solution L, and the microalgae toward the liquid surface of the culturing solution L in the accommodation portion 14.

The culturing gas G, the culturing solution L, and the microalgae rising along the guide flow path 40 are discharged toward the culturing solution L through the discharge port 46. The culturing solution L and the microalgae discharged from the discharge port 46 advance in one direction (X2 direction) in the width direction of the accommodation portion 14 and then descend toward the bottom of the accommodation portion 14. The lowered culturing solution L and microalgae are sucked from the suction port 42 of the guide portion 36. Thereafter, the cycle described above is repeated. Through the cycle, the culturing solution L and the microalgae in the accommodation portion 14 are circulated (stirred).

As for all of the guide portions 36, the discharge ports 46 face in the same direction (X2 direction). Therefore, in the accommodation portion 14, convection is generated in which a flow goes toward the width-direction X2-side end portion and then toward the X1-side end portion in the vicinity of the bottom portion. By this convection, the culturing gas G diffuses throughout the culturing solution L, and the carbon dioxide in the culturing gas G is sufficiently dissolved in the culturing solution L. Convection also stirs the microalgae. As a result, settling or coalescing of microalgae is prevented. For the reasons described above, the microalgae can be well cultured everywhere in the accommodation portion 14.

As described above, the culturing solution L is sulfur-deficient. Further, in the culturing solution L, the amount of dissolved nitrogen is small. Under these circumstances, Chlamydomonas produces hydrogen gas H.

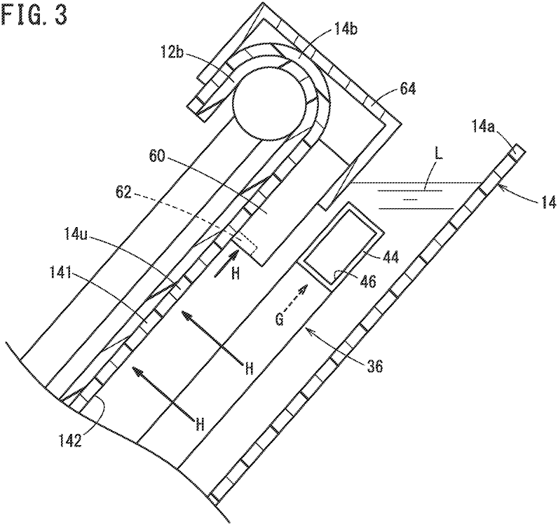

The hydrogen gas H has a smaller specific gravity than the culturing solution L (mainly water W) does. Therefore, as shown in FIG. 3, the hydrogen gas H produced by Chlamydomonas rises toward the side wall 14u of the accommodation portion 14. The hydrogen gas H that has reached the side wall 14u of the accommodation portion 14 moves toward the liquid surface of the culturing solution L along the inner surface 142 of the side wall 14u. In other words, the side wall 14u of the accommodation portion 14 is a deflection portion for changing the progress of the hydrogen gas H and is a guide means for guiding the hydrogen gas H toward the liquid surface.

A gas gathering portion 60 is provided at an upper portion of the side wall 14u. The gathering port 62 of the gas gathering portion 60 is immersed in the culturing solution L. Therefore, the hydrogen gas H moving along the side wall 14u passes through the gathering port 62 and is gathered by the gas gathering portion 60.

While the hydrogen gas H rises in the culturing solution L toward the side wall 14u, the culturing gas G is supplied toward the guide portion 36 in the culturing solution L. Similarly, while the hydrogen gas H moves along the side wall 14u to the gas gathering portion 60, the culturing gas G is supplied toward the guide portion 36 in the culturing solution L. Here, the culturing gas G flows through the guide flow path 40 in the guide portion 36 as described above, and after being discharged from the discharge port 46, convection is generated in the culturing solution L. Therefore, it is prevented that the culturing gas G flows toward the side wall 14u of the accommodation portion 14 together with the hydrogen gas H.

Moreover, the discharge port 46 formed at the upper end of the guide portion 36 is located at the same height as or above the gathering port 62 of the gas gathering portion 60. The specific gravity of the culturing gas G is relatively large. Moreover, the culturing gas G is supplied from the gas supply unit 34 at a predetermined pressure. Therefore, it is not easy for the culturing gas G flowing out from the discharge port 46 to flow around and reach the gathering port 62 of the gas gathering portion 60.

For the reasons described above, the mixing of the culturing gas G with the hydrogen gas H in the culturing solution L is avoided. Therefore, the high-purity hydrogen gas H enters the gas gathering portion 60. Thus, the hydrogen gas H of high purity can be collected.

When bubbles of hydrogen gas H adhere to and remain on the side wall 14u, the operator drives the actuator. Alternatively, the actuator may be driven periodically and automatically. As a result, the two rods 52 rise along the side wall 14u. At this time, since the wiper 50 slides along the inner surface 142 of the side wall 14u, the bubbles of the hydrogen gas H adhering to the side wall 14u are scraped off and move toward the liquid surface. The moved hydrogen gas H is gathered by the gas gathering portion 60 via the gathering port 62 as described above. Thus, the high-purity hydrogen gas H can be efficiently gathered.

Thereafter, the two rods 52 are lowered along the side wall 14u by the actuator. As a result, the wiper 50 returns to the bottom of the accommodation portion 14.

When the hydrogen gas H in the gas gathering portion 60 is collected, the fixing tool 64 is removed from the beam portion 30. As a result, the gas gathering portion 60 is released from the restraint of the fixing tool 64. If necessary, the operator closes the gathering port 62 with a plug and transports the gas gathering portion 60 to the collecting site. Next, the hydrogen gas H gathered by the gas gathering portion 60 is collected in a predetermined collecting tank at a collecting site.

As described above, the collected hydrogen gas H is prevented from being mixed with the culturing gas G. That is, the hydrogen gas H has a relatively high purity. Therefore, it is not particularly necessary to separate the hydrogen gas H from the culturing gas G. As described above, according to the present embodiment, since the separation process is unnecessary, high-purity hydrogen gas H can be easily obtained by a simple procedure. The collected hydrogen gas H is used as a reaction gas for operating a fuel cell, for example. Alternatively, the hydrogen gas H is used as fuel.

After the hydrogen gas H is generated as described above, other useful substances can be obtained from Chlamydomonas. For example, when sulfur is added to culture medium L, Chlamydomonas undergoes photosynthesis. On this basis, carbon dioxide is sufficiently fixed to Chlamydomonas. Thus, carbon dioxide is consumed.

Next, a degreasing treatment is carried out to obtain light oil (so-called biodiesel). Further, if a sugar fermentation process is carried out thereafter, ethanol (so-called bioethanol) can be obtained from the supernatant, and methane can be obtained from the residue.

As described above, the present embodiment discloses the culturing method of gathering gas (H) generated by microalgae in a culturing solution (L), the culturing method including culturing the microalgae while guiding, with a guide portion (36), culturing gas (G) from the bottom of the accommodation portion (14) accommodating the culturing solution and the microalgae toward the liquid surface of the culturing solution, in a state in which the accommodation portion is inclined with respect to the vertical direction, and providing the gas gathering portion (60) at the upper portion of the side wall (14u) of the inclined accommodation portion, with an outer surface (141) of the side wall facing obliquely upward, and the gas generated by the microalgae being cultured is gathered by the gas gathering portion.

Disclosed is a culturing device (10) that gathers gas (H) generated by microalgae in a culturing solution (L), the culturing device including an accommodation portion (14) that accommodates the culturing solution and the microalgae, a support body (16) that supports the accommodation portion in a posture inclined with respect to the vertical direction, a gas supply unit (34) that supplies culturing gas to the culturing solution in the accommodation portion, a guide portion (36) that is provided to the accommodation portion and guides the culturing gas from the bottom of the accommodation portion toward the liquid surface of the culturing solution, and a gas gathering portion (60) that is provided at the upper portion of the side wall of the inclined accommodation portion, the outer surface of the side wall facing obliquely upward, wherein the gas generated by the microalgae being cultured is gathered with the gas gathering portion.

In the present embodiment, the accommodation portion is kept inclined with respect to the vertical direction. In this state, when the microalgae in the accommodation portion generate gas, the gas rises in the culturing solution toward the side wall where the outer surface of the accommodation portion faces obliquely upward. The gas coming into contact with the inner surface of the side wall further moves along the side wall toward the liquid surface of the culturing solution. Here, the gas gathering portion is provided at an upper portion of the side wall. Therefore, the gas moving toward the liquid surface can be gathered by the gas gathering portion.

The culturing gas is supplied to the culturing solution in the accommodation portion. The culturing gas is guided by a guide portion provided to the accommodation portion and moves from the bottom of the accommodation portion toward the liquid surface of the culturing solution. That is, the culturing gas passes through a path different from the path for the gas generated by the microalgae in the accommodation portion. Therefore, mixing of the gas and the culturing gas is prevented.

For the above reasons, the gas produced by the microalgae can be collected as a high-purity gas. This eliminates the need for processes that separate the collected gas and the culturing gas from each other. That is, a high-purity gas can be easily obtained by a simple procedure.

The present embodiment discloses the culturing method wherein the gas gathering portion includes the gathering port (62) opened in the culturing solution, the guide portion includes the discharge port (46) that discharges the culturing gas, and the gathering port is positioned below the discharge port or at the same height as the discharge port.

The present embodiment discloses the culturing device wherein the gas gathering portion includes the gathering port (62) opened in the culturing solution, the guide portion includes the discharge port (46) that discharges the culturing gas, and the gathering port is below the discharge port or at the same height as the discharge port.

It is difficult for the culturing gas discharged from the guide portion to flow around and reach the gathering port located below the upper end of the guide portion or at the same height as the upper end. In other words, the above-described positional relationship further avoids mixing of the culturing gas with the gas produced by the microalgae.

The present embodiment discloses the culturing method wherein the sliding member (50) is provided to the accommodation portion and the sliding member slides along the inner surface (142) of the side wall of the accommodation portion, thereby guiding gas bubbles adhering to the inner surface to the gas gathering portion.

The present embodiment discloses the culturing device provided with a sliding member (50) that is provided to the accommodation portion and slides along the inner surface (142) of the side wall of the accommodation portion.

It is expected that the gas produced by the microalgae may adhere to and stays on the inner surface of the side wall. In this case, when the sliding member is slid along the inner surface of the side wall, the gas adhered to the inner surface is scraped off by the sliding member. Thus, it becomes easy for the gas to separate from the inner surface and move toward the liquid surface. That is, the gas can be efficiently gathered.

The present invention discloses the culturing method wherein Chlamydomonas is used as the microalgae and hydrogen is produced as the gas.

The present invention discloses the culturing device wherein the microalgae is Chlamydomonas and hydrogen is gathered as the gas.

That is, in this case, high-purity hydrogen can be collected. The obtained hydrogen can be used, for example, as a reaction gas for a fuel cell.

It should be noted that the present invention is not limited to the above-described embodiment, and various configurations can be adopted without departing from the gist of the present invention.

For example, a plurality of short gas gathering portions may be used instead of the long gas gathering portion 60 shown in FIG. 1. In this case, a plurality of gas gathering portions may be arranged along the width direction of the accommodation portion 14.

One end of a collecting pipe may be connected to the gas gathering portion 60 and the other end of the collecting pipe may be connected to the collecting tank. In this case, the hydrogen gas H gathered by the gas gathering portion 60 can be collected into the collecting tank via the collecting pipe.

What is claimed is:

1. A culturing method of gathering gas generated by microalgae in a culturing solution, the method comprising
culturing the microalgae while guiding, with a guide portion, culturing gas from a bottom of an accommodation portion accommodating the culturing solution and the microalgae toward a liquid surface of the culturing solution, in a state in which the accommodation portion is inclined with respect to a vertical direction, and
providing a gas gathering portion at an upper portion of a side wall of the inclined accommodation portion, an outer surface of the side wall facing obliquely upward, and gathering, with the gas gathering portion, the gas produced by the microalgae being cultured.

2. The culturing method according to claim 1, wherein
the gas gathering portion includes a gathering port opened in the culturing solution, and
the guide portion includes a discharge port that discharges the culturing gas, wherein the gathering port is positioned below the discharge port or at a same height as the discharge port.

3. The culturing method according to claim 1, wherein the accommodation portion is provided with a sliding member, and the sliding member slides along an inner surface of the side wall of the accommodation portion to thereby guide bubbles of the gas attached to the inner surface to the gas gathering portion.

4. The culturing method according to claim 1, wherein Chlamydomonas is used as the microalgae and hydrogen is produced as the gas.

5. A culturing device that gathers gas generated by microalgae in a culturing solution, the culturing device comprising:
an accommodation portion that accommodates the culturing solution and the microalgae;
a support body that supports the accommodation portion in a posture inclined with respect to a vertical direction;
a gas supply unit that supplies culturing gas to the culturing solution in the accommodation portion;
a guide portion that is provided to the accommodation portion and guides the culturing gas from a bottom portion of the accommodation portion toward a liquid surface of the culturing solution; and
a gas gathering portion that is provided at an upper portion of a side wall of the inclined accommodation portion, an outer surface of the side wall facing obliquely upward, wherein
the gas produced by the microalgae being cultured is gathered with the gas gathering portion.

6. The culturing device according to claim 5,
wherein the gas gathering portion includes a gathering port opened in the culturing solution, and the guide portion includes a discharge port that discharges the culturing gas,
the gathering port is positioned below the discharge port or at a same height as the discharge port.

7. The culturing device according to claim 5, further comprising a sliding member that is provided to the accommodation portion and slides along an inner surface of the side wall of the accommodation portion.

8. The culturing device according to claim 5, wherein Chlamydomonas is used as the microalgae and hydrogen is gathered as the gas.
